(19) United States
(12) Patent Application Publication
(10) Pub. No.: US 2011/0189102 A1
(43) Pub. Date: Aug. 4, 2011

(54) COATED QUANTUM DOTS AND METHODS OF MAKING AND USING THEREOF

(76) Inventors: Brad A. Kairdolf, Lilburn, GA (US); Shuming Nie, Atlanta, GA (US)

(21) Appl. No.: 12/864,763

(22) PCT Filed: Feb. 5, 2009
(86) PCT No.: PCT/US09/33196

§ 371 Date: Sep. 27, 2010

Related U.S. Application Data
(60) Provisional application No. 61/027,103, filed on Feb. 8, 2008.

(51) Int. Cl. H01L 33/06; A61B 5/00; H01L 33/52; B82Y 99/00; B82Y 40/00; B82Y 20/00

(52) U.S. Cl. 424/9.61; 257/13; 438/47; 977/774; 977/890; 977/927; 257/E33.005

(57) ABSTRACT

The present disclosure provides embodiments of a new class of hydroxylated quantum dots. The quantum dots have a hydroxylated coat disposed thereon, and which serves to minimize non-specific cellular binding and to maintain the small size of quantum dot probes. Embodiments of the coated quantum dots of the disclosure are just slightly larger than the diameter of uncoated quantum dots, and are bright with high quantum yields. They are also very stable under both basic and acidic conditions. Embodiments of the hydroxylated quantum dots result in significant reductions in non-specific binding relative to that of carboxylated dots, and to protein and PEG-coated dots. Embodiments of the disclosure are advantageous in a range of biological applications where non-specific binding is a major problem, such as in multiplexed biomarker staining in cells and tissues, detection of biomarkers in body fluid samples (blood, urine, etc.), as well as live cell imaging.

### CROSS-REFERENCE TO RELATED APPLICATIONS

This application claims priority to U.S. Provisional Patent Application Ser. No. 61/027,103, entitled “Coated Quantum Dots and Methods of Use” filed on Feb. 8, 2008, the entirety of which is hereby incorporated by reference.

### STATEMENT REGARDING FEDERALLY SPONSORED RESEARCH OR DEVELOPMENT

This invention was made with government support under NIH Grants Nos. P20 GM072069, RO1 CA108468, and awarded by the U.S. National Institutes of Health of the United States government. The government has certain rights in the invention.

### FIELD OF THE DISCLOSURE

The present disclosure relates generally to nanostructures, and relates more particularly to coated nanostructures.

### BACKGROUND

Semiconductor quantum dots (quantum dots) are a new class of fluorescent labeling agents and have recently been used for a broad range of biological applications (Bruchez et al., (1998) Science 281: 2013-2016; Chan et al., (1998) Science 281: 2016; Wu et al., (2003) Nat. Biotechnol. 21: 41-46; Dubertret et al., (2002) Science 298: 1759-1762; Gao et al., (2004) Nat. Biotechnol. 22: 969-976; Kim et al., (2004) Nat. Biotechnol. 22: 93-97; Alivisatos, P. (2004) Nat. Biotechnol. 22: 47-52; Chattopadhyay et al., (2006) Nat. Med. 12: 972-977; Medintz et al., (2005) Nat. Mater. 4: 435-446; Smith et al., (2004) Photochem. Photobiol. 80: 377-385; Michalet et al., (2005) Science 307: 538-544). This broad interest is driven by their unique optical and electronic properties such as size-tunable light emission, superior signal brightness, resistance to photobleaching, and simultaneous excitation of multiple fluorescence colors (Alivisatos, P. (2004) Nat. Biotechnol. 22: 47-52; Chattopadhyay et al., (2006) Nat. Med. 12: 972-977; Medintz et al., (2005) Nat. Mater. 4: 435-446; Smith et al., (2004) Photochem. Photobiol. 80: 377-385). Recent advances have led to highly bright and stable quantum dot probes that are well suited for multiplexed molecular profiling of intact cells and clinical tissue specimens (Matsuno et al., (2005) J. Histochem. Cytochem. 53: 833-838; Xing et al., (2007) Nat. Protoc. 2: 1152-1165; Yezhelyev et al., (2006) Lancet Oncol., 7: 657-667; Xing et al., (2006) Int. J. Nanomedicine, 1: 473-481; Gao et al., (2005) Curr. Opin. Biotechnol. 16: 63-72; Yezhelyev et al., (2007) Adv. Mater. 19: 3146-3151; Ness et al., (2003) J. Histochem. Cytochem. 51: 981-987).

In contrast to in vivo clinical imaging where the potential toxicity of cadmium-containing quantum dots is a major concern, histological and cellular staining is performed on in vitro or ex vivo clinical patient samples. As a result, the use of multicolor quantum dot probes for cellular staining is likely one of the most important and clinically relevant applications in the near term (Xing et al., (2007) Nat, Protoc. 2: 1152-1165; Yezhelyev et al., (2006) Lancet Oncol. 7: 657-667; Xing et al., (2006) Int. J. Nanomedicine 1: 473-481). However, a major problem is that quantum dot probes tend to be “sticky” and often bind non-specifically to cellular membranes, proteins, and extracellular matrix materials. In particular, nanoparticles with highly charged surface groups such as carboxylic acids and amines have been shown to exhibit strong non-specific binding to various cells and tissues (Pathak et al., (2001) J. Am. Chem. Soc. 123: 4103-4104; Gerion et al., (2001) J. Am. Chem. Soc. 124: 24; Bentzen et al., (2005) Bioconjugate Chem., 16: 1488-1494; Duan & Nie (2007) J. Am. Chem. Soc. 129: 3333-3338). This non-specific binding problem causes a high level of background fluorescence that degrades the signal-to-noise ratio and limits tagging specificity and detection sensitivity.

A number of surface encapsulation methods have been used for quantum dot solubilization and bioconjugation, including direct ligand-exchange reactions and indirect surface encapsulation using silica, lipids, and amphiphilic polymers (Bruchez et al., (1998) Science 281: 2013-2016; Chan et al., (1998) Science 281: 2016; Wu et al., (2003) Nat. Biotechnol. 21: 41-46; Dubertret et al., (2002) Science 298: 1759-1762; Duan & Nie (2007) J. Am. Chem. Soc. 129: 3333-3338; Zhelev et al., (2006) J. Am. Chem. Soc. 128: 6324-6325; Uyeda et al., (2005) J. Am. Chem. Soc. 127: 3870-3878; Wang et al., (2002) J. Am. Chem. Soc. 124: 2293-2298). To reduce non-specific binding, quantum dots are often attached to polyethylene glycol (PEG) (Wu et al., (2003) Nat. Biotechnol. 21: 41-46; Bentzen et al., (2005) Bioconjugate Chem. 16: 1488-1494), a nontoxic and hydrophilic polymer that is commonly used to improve drug biocompatibility and systemic circulation (Couvreur & Vauthier, (2006) Pharm. Res. 23: 1417-1450; Torchilin, V. P. (2007) Pharm. Res. 24: 1-16; Moghimi et al., (2001) Pharmacol. Rev. 53: 283-318; Duncan, R. (2006) Nat. Rev. Cancer 6: 688-701). Pegylated quantum dots have nearly neutral surface charges and are able to maintain colloidal stability under various experimental conditions.

Although recent work has successfully used pegylated quantum dots for both in vitro (Wu et al., (2003) Nat. Biotechnol. 21: 41-46; Bentzen et al., (2005) Bioconjugate Chem. 16: 1488-1494) and in vivo (Dubertret et al., (2002) Science 298: 1759-1762; Gao et al., (2004) Nat. Biotechnol. 22: 969-976; Kim et al., (2004) Nat. Biotechnol. 22: 93-97; Ballou et al., (2004) Bioconjugate Chem. 15: 79-86) applications, non-specific binding to complex intracellular and extracellular materials is still a bottleneck in improving detection sensitivity and specificity (Xing et al., (2007) Nat. Protoc. 2: 1152-1165). In addition, PEG-coated particles have larger hydrodynamic diameters than the corresponding uncoated particles, which can prevent the probes from accessing biological targets deep within complex tissue and cellular structures.

### SUMMARY

The present disclosure provides a new class of nanoparticle hydroxylated quantum dots. The quantum dots have a hydroxylated coat disposed thereon that serves to minimize non-specific cellular binding while retaining the small size of quantum dot probes. Embodiments of the present disclosure were prepared from carboxylated (—COOH) dots via a hydroxylation step. Optional cross-linking within the coating is also possible. Embodiments of the hydroxyl-coated dots have compact sizes of about 13 to about 14 nm hydrodynamic diameter, just slightly larger than the diameter of uncoated quantum dots, and are bright with about 65% quantum yields. Embodiments of the present disclosure are also very stable under both basic and acidic conditions. Quantitative data from human cancer cells indicate that the hydroxylated quantum dots results in a significant (>100-fold) reduction in non-specific binding relative to that of carboxylated dots, and a smaller, but still significant, reduction relative to protein and PEG-coated dots. The data indicate that surface charge plays a significant role in the non-specific binding of these nanoparticles to cellular components. The nanoparticles of the disclosure are advantageous in a range of biological applications where non-specific binding is a major problem, such as in multiplexed biomarker staining in cells and tissues, detection of biomarkers in body fluid samples (blood, urine, etc.), as well as live cell imaging.

One aspect of the present disclosure provides a nanostructure, comprising: a quantum dot; a hydrophobic layer disposed on the quantum dot; and a coat disposed on said hydrophobic layer, wherein the coating has a substantially hydroxylated outer surface or a substantially zwitterion outer surface.

In preferred embodiments of the present disclosure, the nanostructure has a coat, wherein said coat has a substantially hydroxylated outer surface.

In the various embodiments of the present disclosure, the nanostructure may have substantially no detectable non-specific cellular binding compared to a nanostructure having a coat that is not substantially hydroxylated and at the same concentration.

Another aspect of the disclosure provides methods of synthesizing a nanostructure, where the methods comprise: (a) providing a quantum dot, wherein the quantum dot comprises a hydrophobic layer thereon; (b) encapsulating the quantum dot by contacting the quantum dot with a polymer comprising a multiplicity of carboxyl groups; and (c) replacing a preponderance of the carboxyl groups with a multiplicity of hydroxyl groups.

Yet another aspect of the disclosure provides methods imaging, comprising: providing a nanostructure of the present disclosure; administering the nanostructure to a recipient host; and imaging the recipient host, whereby the nanostructure delivered to the recipient host provide an image of a tissue of the recipient host, and wherein the image has a substantially reduced non-tissue-specific background fluorescence when compared to an image generated with a nanostructure not having a substantially hydroxylated coat.

### BRIEF DESCRIPTION OF THE DRAWINGS

Further aspects of the present disclosure will be more readily appreciated upon review of the detailed description of its various embodiments, described below, when taken in conjunction with the accompanying figures.

FIG. 1A illustrates diagrammatically the surface coating chemistry and structures of polymer-encapsulated (CdSe/CdS/ZnS) quantum dots. The schematic diagram shows the conversion of carboxylated quantum dots (coated with polyacrylic acid octylamine) to hydroxylated and cross-linked quantum dots. The small-molecule hydroxylation agent is 1,3-diamino-2-propanol (DAP).

FIG. 1B illustrates diagrammatically the surface coating structure of polymer-encapsulated (CdSe/CdS/ZnS) quantum dots hydroxylated, but not cross-linked, quantum dots.

FIG. 1C is a digital transmission electron micrograph showing the structure of encapsulated quantum dots after surface hydroxylation and cross-linking.

FIGS. 2A-2C illustrate graphs of UV-Vis absorption (left sloping curves) and fluorescence emission spectra (symmetric peaks at 640 nm). FIG. 2A, hydrophobic quantum dots in chloroform; FIG. 2B, solubilized quantum dots in buffer solution; and FIG. 2C, hydroxylated quantum dots in buffer solution.

FIG. 3A is a graph showing the hydrodynamic diameter data obtained from hydroxylated quantum dots, carboxylated quantum dots, streptavidin-coated quantum dots, QTracker quantum dots, and antibody-conjugated quantum dots by using dynamic light scattering measurements.

FIG. 3B is a digital image of a gel electrophoretic analysis corresponding to hydroxylated quantum dots, carboxylated quantum dots, streptavidin coated quantum dots, QTracker quantum dots, and antibody-conjugated quantum dots.

FIGS. 4A-4D illustrate a series of digital fluorescence microscopy images of hydroxylated and carboxylated quantum dots non-specifically bound to fixed human HeLa cells. FIGS. 4A and 4B: carboxylated quantum dots with (FIG. 4A) and without (FIG. 4B) DAPI counter staining of cell nuclei, showing intense non-specific cellular binding. (FIGS. 4C and 4D): hydroxylated quantum dots with (FIG. 4C), and without (FIG. 4D), DAPI counter staining of cell nuclei, showing the absence of non-specific cellular binding.

FIGS. 5A and 5B are graphs illustrating the quantitative evaluation and comparison of non-specific cellular binding for various quantum dot surface coatings. FIG. 5A is a bar graph illustrating normalized fluorescence staining at 20 nM quantum dot concentration, as measured by microplate assays. FIG. 5B is a graph of plots of non-specific cellular binding signal intensities as a function of quantum dot concentration.

FIG. 6A is a digital image of a gel electrophoretic analysis of quantum dots with increasing degrees of hydroxylation, from approximately 100% carboxylation (left) to approximately 100% hydroxylation (right).

FIG. 6B is a bar graph illustrating the non-specific cellular binding data for quantum dots with increasing degrees of hydroxylation, from 100% —COOH (left) to 100% —OH (right).

The figures are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

The term “quantum dot” (quantum dots) as used herein refers to semiconductor nanocrystals or artificial atoms, which are semiconductor crystals that contain anywhere between 100 to 1,000 electrons and range from about 2 to about 10 nm. Some quantum dots can be between about 10 to about 20 nm in diameter. Quantum dots have high quantum yields, which makes them particularly useful for optical applications. Quantum dots are fluorophores that fluoresce by forming excitons, which can be thought of as the excited state of traditional fluorophores, but which have much longer lifetimes of up to 200 nanoseconds. This property provides quantum dots with low photobleaching. The energy level of quantum dots can be controlled by changing the size and shape of the quantum dot, and the depth of the quantum dots' potential. One of the optical features of small excitonic quantum dots is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the quantum dot. Larger quantum dots have more energy levels which are more closely spaced, thus allowing the quantum dot to absorb photons containing less energy, i.e. those closer to the red end of the spectrum. Because the emission frequency of a dot is dependent on the bandgap, it is therefore possible to control the output wavelength of a dot with extreme precision. Colloidally prepared quantum dots are free floating and can be attached to a variety of molecules via metal coordinating functional groups. These groups include but are not limited to thiol, amine, nitrile, phosphine, phosphine oxide, phosphonic acid, carboxylic acids or other ligands. By bonding appropriate molecules to the surface, the quantum dots can be dispersed or dissolved in nearly any solvent or incorporated into a variety of inorganic and organic films.

The term “carbodiimide” as used herein refers to a class of organic substances comprising a R—N═C═N—R′ moiety. The R and R′ groups may be any organic radicals. For example, when R and R′ are cyclohexyl groups, the carbodiimide is 1,3-dicyclohexylcarbodiimide, a dehydrating reagent well known in the art. A water-soluble carbodiimide is a carbodiimide that has sufficient solubility in water to form a homogeneous solution. Typically, a water-soluble carbodiimide contains an ionic group, such as an ammonium salt, to confer water-solubility upon the molecule. The water-soluble carbodiimides include, but are not limited to, 1-ethyl-3-(3-dimethylaminopropyl) carbodiimide (ECDI), N-(3-dimethylaminopropyl)-N′-ethylcarbodiimide hydrochloride (EDAC), N-Cyclohexyl-N′-(2-morpholinoethyl)carbodiimide metho-p-toluenesulfonate, and 1-ethyl-3-(4-azonia-4,4-dimethylpentyl) carbodiimide.

The term “amine alcohol” as used herein refers to a compound having at least one amine group and at least one alcohol group and may include such compounds as, but not limited to, 1,3-diamino-2-propanol (DAP), ethanolamine, 3-amino-1-propanol, 3-amino-1,2-propanediol, 2-amino-1,3-propanediol (serinol), 4-amino-1-butanol, 2-(2-aminoethoxy)ethanol, Tris(hydroxymethyl)aminomentane, 1,4-diamino-2,3-butanediol, 5-amino-1-pentanol, 2-(3-aminopropylamino)ethanol, 6-amino-1-hexanol, and n,n-bis(2-hydroxyethyl)ethylenediamine.

The term “alkylamine” as used herein refers to an alkyl chain having at least one, and preferably two amine groups thereon, where the alkylamine may be selected from, but not limited to, (2-aminoethyl)trimethylammonium chloride hydrochloride, n,n-dimethylethylenediamine, 3-(dimethylamino)-1-propylamine, 2-(aminomethyl)-2-methyl-1,3-propanediamine trihydrochloride, n-(2-aminoethyl)-1,3-propanediamine, and 3,3′-diamino-N-methyldipropylamine.

By “administration” is meant introducing an embodiment of the present disclosure into a recipient host. Administration can include routes, such as, but not limited to, intravenous, oral, topical, subcutaneous, intraperitoneal, intraarterial, inhalation, vaginal, rectal, nasal, introduction into the cerebrospinal fluid, or instillation into body compartments can be used.

The term “host” or includes humans, mammals (e.g., cats, dogs, horses, etc.), living cells, and other living organisms. A living organism can be, for example, a single eukaryotic cell or as complex as a mammal. Hosts to which embodiments of the present disclosure may be administered can be mammals, particularly primates, especially humans. Veterinary applications will be, e.g., livestock: cattle, sheep, goats, cows, swine, and the like; poultry: chickens, ducks, geese, turkeys, and the like; and domesticated animals pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications.

The term “cancer”, as used herein shall be given its ordinary meaning and is a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Discussion

Quantum-dot (quantum dot) nanocrystals are promising fluorescent probes for multiplexed staining assays in biological applications. However, non-specific quantum dot binding to cellular membranes and proteins remains a limiting factor in detection sensitivity and specificity. Embodiments of the present disclosure encompasses hydroxyl (—OH) coated quantum dots for minimizing non-specific cellular binding and for substantially overcoming the bulk size problems associated with other types of surface coatings. Embodiments of the hydroxylated quantum dots of the present disclosure may be prepared from carboxylated (—COOH) dots via a hydroxylation and cross-linking process. With a compact hydrodynamic diameter of about 13 to about 14 nm, they are highly fluorescent (>60% quantum yields) and are stable under basic and/or acidic conditions. Using human cancer cells, their non-specific binding properties were evaluated against that of carboxylated, protein coated, and polyethylene glycol (PEG)-coated quantum dots. Quantitative cellular staining data indicated that the hydroxylated quantum dots of the present disclosure result in a significant reduction in non-specific binding relative to that of carboxylated dots, and a more moderate, but still significant, reduction relative to PEG- and protein-coated dots.

The hydrophobic protection structure may include a capping ligand layer and/or a copolymer layer (e.g., amphiphilic block copolymer). The following illustrative examples will use amphiphilic block copolymers, but other copolymers such as, but not limited to, amphiphilic random copolymers, amphiphilic alternating copolymers, amphiphilic periodic copolymers, and combinations thereof, can be used in combination with block copolymers, as well as individually or in any combination. In addition, the term “amphiphilic block copolymer” will be termed “block copolymer” hereinafter.

The nanostructure can include a number of types of nanoparticle such as, but not limited to, a semiconductor nanoparticle. In particular, semiconductor quantum dots suitable for use in the nanostructures of the present disclosure are described in more detail below and in U.S. Pat. No. 6,468,808 and International Patent Application WO 03/003015, which are incorporated herein by reference.

The nanostructure can include quantum dots such as, but not limited to, luminescent semiconductor quantum dots. In general, quantum dots include a core and a cap, however, uncapped quantum dots can be used as well. The “core” is a nanometer-sized semiconductor. While any core of the IIA-VIA, IIIA-VA or IVA-IVA, IVA-VIA semiconductors can be used in the context of the present disclosure, the core must be such that, upon combination with a cap, a luminescent quantum dot results. A IIA-VIA semiconductor is a compound that contains at least one element from Group IIB and at least one element from Group VIA of the periodic table, and so on. The core can include two or more elements. In one embodiment, the core is a IIA-VIA, IIIA-VA or IVA-IVA semiconductor that ranges in size from about 1 nm to about 20 nm. In another embodiment, the core is more preferably a IIA-VIA semiconductor and ranges in size from about 2 nm to 10 nm. For example, the core can be CdS, CdSe, CdTe, ZnSe, ZnS, PbS, PbSe or an alloy.

The “cap” is a semiconductor that differs from the semiconductor of the core and binds to the core, thereby forming a surface layer on the core. The cap can be such that, upon combination with a given semiconductor core a luminescent quantum dot results. The cap should passivate the core by having a higher band gap than the core. In one embodiment, the cap is a IIA-VIA semiconductor of high band gap. For example, the cap can be ZnS or CdS. Combinations of the core and cap can include, but are not limited to, the cap is ZnS when the core is CdSe or CdS, and the cap is CdS when the core is CdSe. Other exemplary quantum does include, but are not limited to, CdS, ZnSe, CdSe, CdTe, CdSeₓTe₁₋ₓ, InAs, InP, PbTe, PbSe, PbS, HgS, HgSe, HgTe, CdHgTe, and GaAs.

The wavelength emitted (i.e., color) by the quantum dots can be selected according to the physical properties of the quantum dots, such as the size and the material of the nanocrystal. Quantum dots are known to emit light from about 300 nanometers (nm) to about 1700 nm (e.g., UV, near IR, and IR). The colors of the quantum dots include, but are not limited to, red, blue, green, and combinations thereof. The color or the fluorescence emission wavelength can be tuned continuously. The wavelength band of light emitted by the quantum dot is determined by either the size of the core or the size of the core and cap, depending on the materials which make up the core and cap. The emission wavelength band can be tuned by varying the composition and the size of the quantum dot and/or adding one or more caps around the core in the form of concentric shells.

The intensity of the color of the quantum dots can be controlled. For each color, the use of 10 intensity levels (0, 1, 2, . . . 9) gives 9 unique codes (10¹-1), because level “0” cannot be differentiated from the background. The number of codes increase exponentially for each intensity and each color used. For example, a three color and 10 intensity scheme yields 999 (10³-1) codes, while a six color and 10 intensity scheme has a theoretical coding capacity of about 1 million (10⁶-1). In general, n intensity levels with m colors generate (n^{m}-1) unique codes. Use of the intensity of the quantum dots has applications in nanostructures including a plurality of different types of quantum dots having different intensity levels and also in nanostructures including a plurality of different types of quantum dots having different intensity levels that are included in a porous material. The quantum dots are capable of absorbing energy from, for example, an electromagnetic radiation source (of either broad or narrow bandwidth), and are capable of emitting detectable electromagnetic radiation at a narrow wavelength band when excited. The quantum dots can emit radiation within a narrow wavelength band (FWHM, full width at half maximum) of about 40 nm or less, thus permitting the simultaneous use of a plurality of differently colored quantum dots with little or no spectral overlap.

The synthesis of quantum dots is well known and is described in U.S. Pat. Nos. 5,906,670; 5,888,885; 5,229,320; 5,482,890; 6,468,808; 6,306,736; 6,225,198, etc., International Patent Application WO 03/003015, (all of which are incorporated herein by reference in their entireties) and in many research articles. The wavelengths emitted by quantum dots and other physical and chemical characteristics have been described in U.S. Pat. No. 6,468,808 and International Patent Application WO 03/003015, both of which are incorporated herein by reference in their entireties.

As mentioned above, the hydrophobic protection structure of the nanostructures according to the present disclosure includes the capping ligand and/or the block copolymer. In particular, when the nanoparticle is a quantum dot, the hydrophobic protection layer may include the capping ligand and a block copolymer, where the capping ligand and the block copolymer interact with one another to form the hydrophobic protection structure. As such, the capping ligand and the block copolymer are selected to form an appropriate hydrophobic protection structure. For example, the block copolymer and the nanoparticle can interact through interactions such as, but not limited to, hydrophobic interactions, hydrophilic interactions, pi-stacking, etc., depending on the surface coating of the nanoparticle and the molecular structure of polymers.

The capping ligand caps the nanoparticle (e.g., quantum dot) and forms a layer on the nanoparticle, which subsequently bonds with a copolymer to form the hydrophobic protection structure. The capping ligand can include compounds such as, but not limited to, an O═PR₃ compound, an O═PHR₂ compound, an O═PHR₁ compound, a H₂NR compound, a HNR₂ compound, a NR₃ compound, a HSR compound, a SR₂ compound, and combinations thereof. “R” can be a C₁ to C₁₈ hydrocarbon, such as but not limited to, linear hydrocarbons, branched hydrocarbons, cyclic hydrocarbons, substituted hydrocarbons (e.g., halogenated), saturated hydrocarbons, unsaturated hydrocarbons, and combinations thereof. Preferably, the hydrocarbon is a saturated linear C₄ to C₁₈ hydrocarbon, a saturated linear C₆ to C₁₈ hydrocarbon, and a saturated linear C₁₈ hydrocarbon. A combination of R groups can be attached to P, N, or S. In particular, the chemical can be selected from tri-octylphosphine oxide, stearic acid, and octyldecyl amine.

In embodiments, the quantum dot can be overcoated with a polymer, through interactions such as, but not limited to, hydrophobic interactions, hydrophilic interactions, covalent bonding, and the like. In an embodiment, the coat (also referred to as “coating”) can include a amphiphilic polymer coat. For example, the amphiphilic copolymers include hydrophobic blocks and hydrophilic blocks. The amphiphilic copolymer includes, but is not limited to, amphiphilic block copolymers, amphiphilic random copolymers, amphiphilic alternating copolymers, amphiphilic periodic copolymers, and combinations thereof.

The thickness of each layer disposed on the quantum dot can vary significantly depending on the particular application. In general, the thickness is about 0.5 to about 20 nm, about 0.5 to about 15 nm, about 0.5 to about 10 nm, and about 0.5 to about 5 nm.

Therapeutic agents, biological compounds (e.g., a protein, an antibody, a polynucleotide, a polypeptide, and an aptamer), linkers, and/or other compounds can be attached directly to the nanoparticle and/or attached to the polymer layer disposed on the nanoparticle. In addition, a therapeutic agent, a biological compound, a linker, and/or other compounds can be attached indirectly to the nanoparticle and/or attached to the polymer layer disposed on the nanoparticle. For example, the therapeutic agent and/or biological compound can be attached in series via one or more linkers.

The therapeutic agents, the biological compounds, the linkers, and/or other compounds, can be linked to the nanoparticle using any stable physical and/or chemical association to the nanoparticle directly or indirectly by any suitable means. For example, the component can be linked to the nanoparticle using a covalent link, a non-covalent link, an ionic link, and a chelated link, as well as being absorbed or adsorbed onto the nanoparticle. In addition, the component can be linked to the nanoparticle through hydrophobic interactions, hydrophilic interactions, charge-charge interactions, π-stacking interactions, combinations thereof, and like interactions.

The linker can include a functional group (e.g., an amine group) on the layer disposed on the quantum dot and/or the linker can include a separate compound attached to the quantum dot or the layer at one end and the protein, the antibody, the polynucleotide, the polypeptide, the aptamer, the linker, other compounds, or another linker at the other end. The linker can include functional groups such as, but not limited to, amines, carboxylic acids, hydroxyls, thios, and combinations thereof. The linker can include compounds such as, but not limited to, DTPA, EDTA, DOPA, EGTA, NTA, and combinations thereof. In an embodiment, the linker and the chelator compound are the same, but in other embodiments they can be different. The percentage of linkers attached to the chelator compound, contrast agent, and/or another linker can be about 0.1 to about 100%.

The embodiments of the present disclosure encompass nanostructures comprising a quantum dot that may further include at least one other layer or component selected from, but not limited to, such as a capping layer, a polymer layer, a target-specific probe, or any combination thereof, and a coating layer modified to have a preponderance of hydroxyl groups at the outside surface of the nanostructure. The hydroxyl groups, provided by conversion of such as carboxyl groups, provide nanostructures with a significantly reduced ability to non-specifically bind to biological molecules or structures such, but not limited to, cell surfaces when compared to similar nanostructures not having the hydroxyl coat of the present disclosure. The non-specific binding can be reduced to levels that are barely detectable, if at all. The nanostructures of the disclosure, when conjugated to a target-specific probe such as, but not limited to, an antigen-specific antibody, a receptor specific ligand and the like, and then delivered to a recipient host, can provide greatly enhanced imaging of the targeted structure due to the reduction in the non-specific background fluorescence. The present disclosure, therefore, provides coated quantum dot nanostructures useful as imaging agents. It is also contemplated that within the scope of the present disclosure are delivery systems where a compound to be delivered to a targeted cell or tissue may also be monitored by the quantum dot fluorescence enhanced by the coatings of the disclosure.

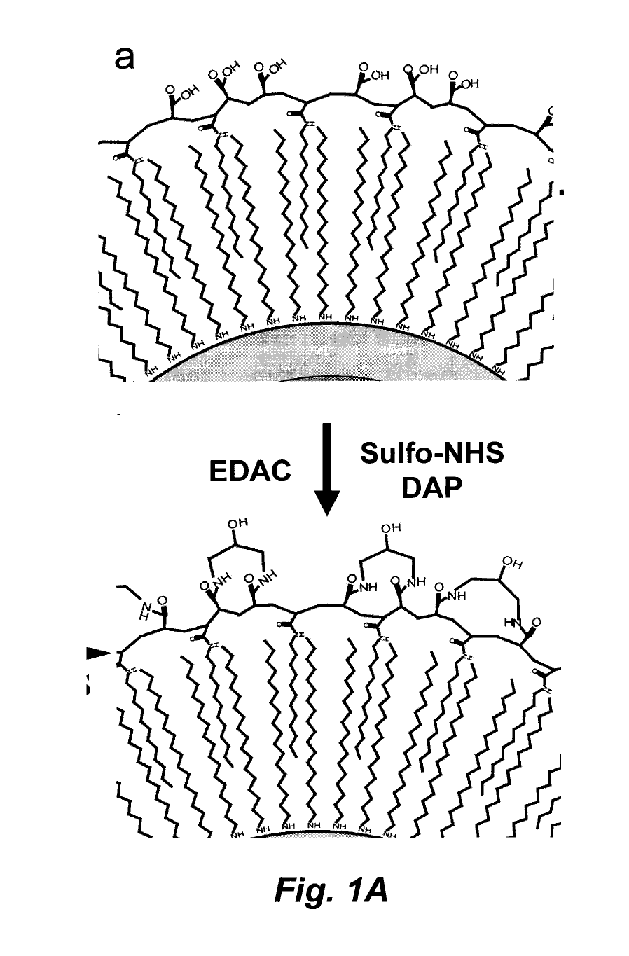

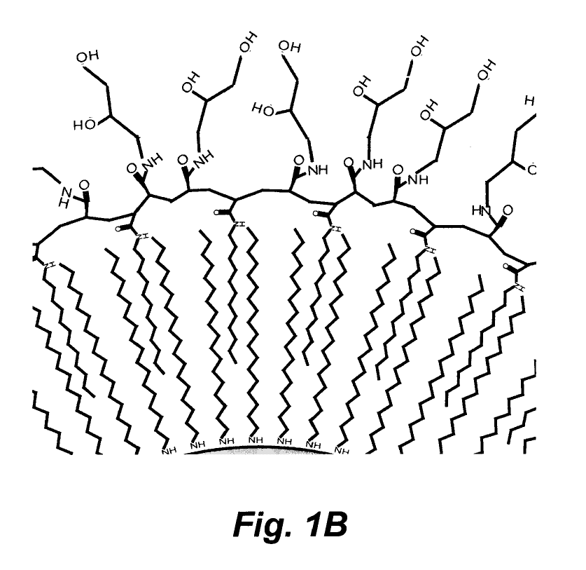

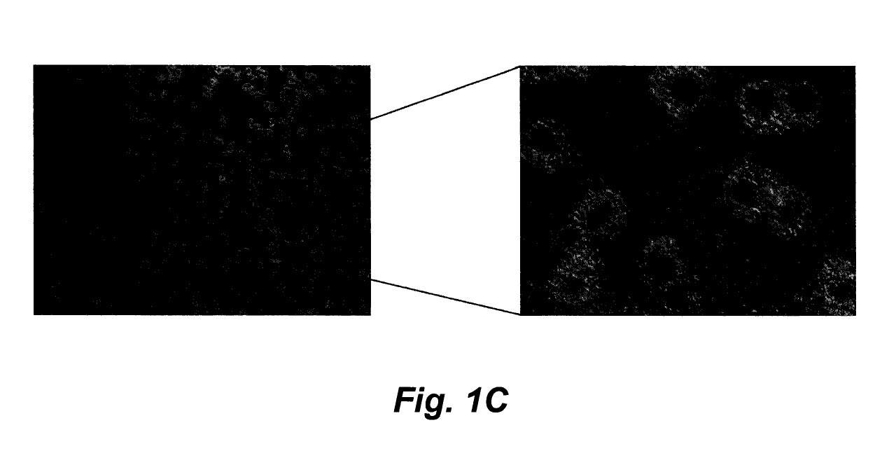

Now having described the embodiments of the nanostructure according to the disclosure in general, the following are non-limiting illustrative examples of embodiments of the nanostructures, methods of making, and uses thereof, of the present disclosure. One skilled in the art would understand that many experimental conditions can be modified, but it is intended that these modifications be within the scope of the embodiments of the present disclosure. Surface Coating Chemistry. Referring now to the schemes shown in FIGS. 1A and 1B, carboxylic acid functional groups can be modified by the methods of the present disclosure with a small hydroxyl-containing molecule (such as, but not limited to, 1,3-diamino-2-propanol or DAP) to yield quantum dots with hydroxyl functional groups on the surface. Based on a structural model of the polymer and the quantum dot surface area, it is estimated that each quantum dot can be covered with about 150 amphiphilic polymer molecules, leading to approximately 2500 carboxylic acid groups (each polymer molecule has approximately fifteen COOH groups) potentially available for conversion. These COOH groups can then be converted to OH groups by the hydroxylation and optional cross-linking process of the disclosure, thereby, creating a cage-like shell that locks the polymer coating in place. The hydroxylated quantum dots are stable for at least 6 months in borate buffer solution at 4° C. They show no aggregation in acidic environments, which has been a problem for traditional quantum dots with exposed carboxylic acid functional groups (due to protonation at low pHs), as reported previously by Matsuno et al., (2005) J. Histochem. Cytochem. 53: 833-838. Transmission electron microscopy (TEM) clearly shows polymer-encapsulated quantum dots with an average diameter of about 13 nm. Fluorescence microscopic imaging further reveals a characteristic blinking behavior for immobilized quantum dots on a glass slide, a property discussed in Nirmal et al., (1996) Nature 383: 802-804, incorporated herein by reference in its entirety, that indicates that the dots are primarily well dispersed single particles.

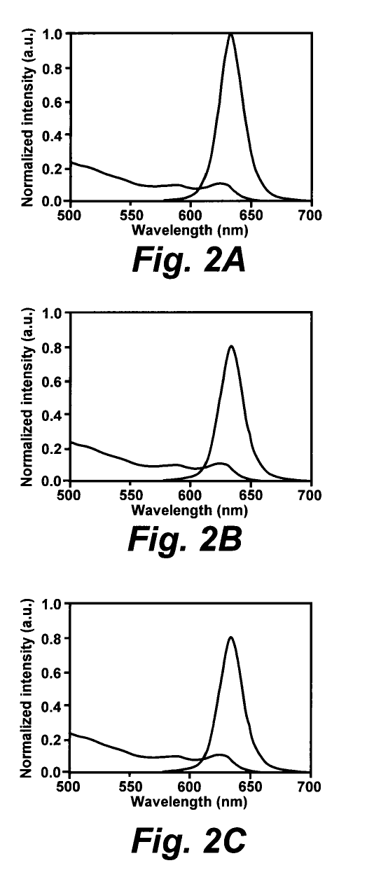

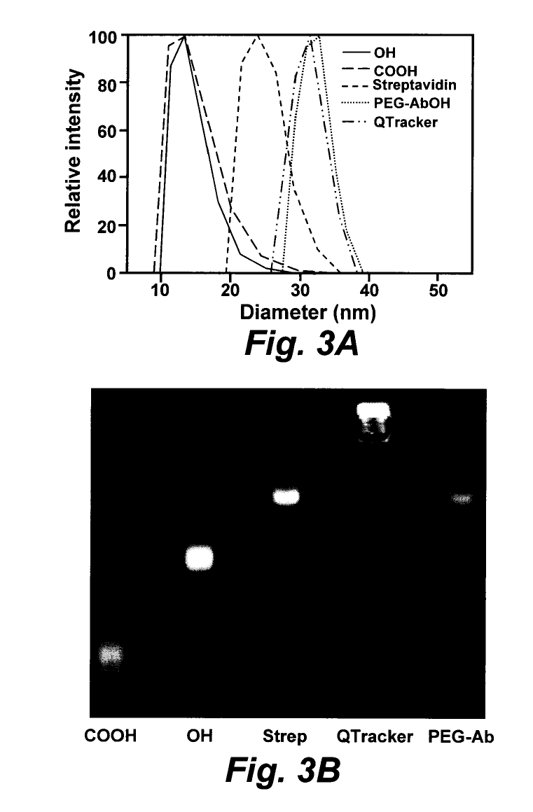

QD Size, Charge, and Optical Properties. As shown in FIGS. 2A-2C, nanostructure polymer encapsulation and subsequent hydroxylation by the methods of the present disclosure have no significant effects on the quantum dot's optical properties such as UV-Vis absorption and fluorescence emission. The water-solubilized quantum dots and the hydroxylated quantum dots have nearly identical fluorescence emission spectra with a quantum yield of about 65% and a spectral width (full width at half maximum or FWHM) of about 23 nm. This surface treatment also has little or no effect on the overall particle size as measured by dynamic light scattering (DLS). In fact, the hydrodynamic diameters (of about 13 nm to about 15 nm) of the hydroxylated dots are approximately the same, or even slightly smaller, than that of the carboxylated dots (of about 14 nm to about 16 nm). Although surface hydroxylation can be expected to slightly increase the overall particle size, this process also reduces the particle surface charge and the electrical double layer thickness and, therefore, the hydrodynamic radius. In contrast, quantum dots coated with PEG and/or proteins often have hydrodynamic diameters of between about 25 to about 30 nm, i.e. about twice the size of hydroxylated dots of the present disclosure as shown, for example, in FIGS. 3A and 3B. The decrease in surface charge after hydroxyl modification was further supported by zeta potential and gel electrophoresis measurements. Quantum dots with carboxylic acid surface groups have a measured zeta potential of about −40 mV at pH 8.5, comparable to values reported previously (Smith et al., (2006) Phys. Chem. Chem. Phys. 8: 3895-3903). The hydroxylated quantum dots of the present disclosure show a significant decrease in surface charge, with a zeta potential of about −20 mV at pH 8.5.

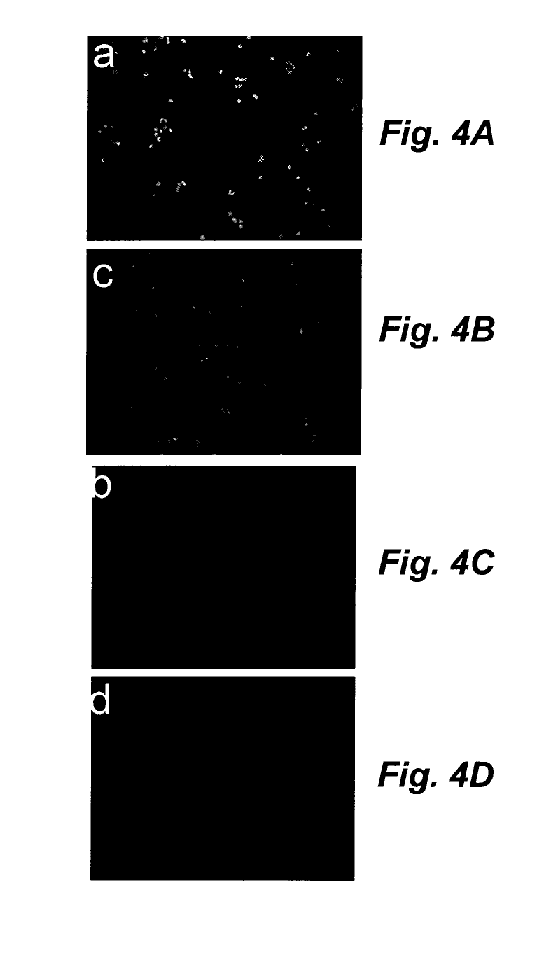

For gel electrophoresis measurements, quantum dots coated with a PEG layer were expected to migrate very slowly due to their large sizes and more neutral zeta potentials. Likewise, streptavidin-conjugated dots may be expected to migrate slowly, again because of their large sizes and reduced charges due to protein shielding. Quantum dots with carboxylic acid surface groups were expected to migrate most rapidly towards the positive electrode because of their small sizes and high negative charges. In comparison, the hydroxylated quantum dots of the present disclosure would migrate more slowly than carboxylated quantum dots due to their reduced surface charges. Gel electrophoresis studies revealed that carboxylated quantum dots migrated the farthest in distance, in agreement with their strongly negative zeta potential and small size, as shown in FIG. 3B. The hydroxylated quantum dots migrate less than the carboxylic acid quantum dots, but more than the protein or PEG-coated dots, probably due to their smaller size. Streptavidin- and secondary antibody-conjugated quantum dots showed a slow migration toward the positive electrode, suggesting a net negative surface charge. This negative charge suggested that that the antibody-conjugated quantum dots are sparsely coated with PEG since heavy pegylation would produce nanoparticle with nearly neutral zeta potentials (Smith et al., (2006) Phys. Chem. Chem. Phys. 8: 3895-3903). Evaluation of Nonspecific Cellular Binding. Human cancer cells were used to compare the non-specific binding properties of quantum dots with different surface coatings. As illustrated in FIGS. 4A-4D, for example, carboxylated quantum dots show very high non-specific cellular binding when incubated at a concentration of 20 nM; significant non-specific binding is also observed at quantum dot concentrations lower than 2 nM. In contrast, there is no detectable non-specific binding for the hydroxylated quantum dots of the present disclosure at similar concentrations. DAPI nuclear staining was used to ensure an equivalent cell density between the samples.

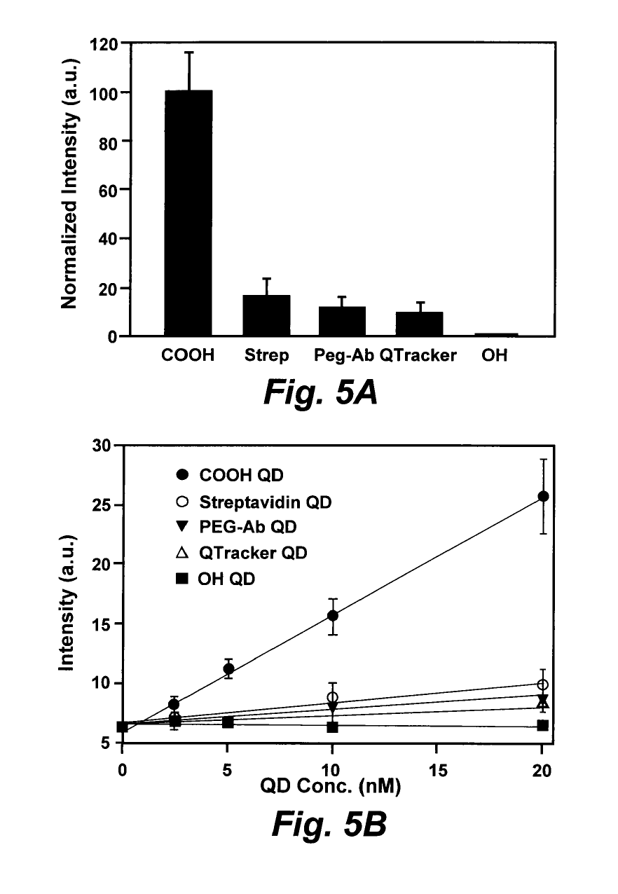

The quantum dot concentration could be increased to 100 nM to determine if any non-specific binding could be detected for the hydroxyl modified quantum dots. Even at this high concentration, there was minimal non-specific binding relative to the negative control. For quantitative and statistical analysis, a fluorescence microplate assay was used to measure a large population of quantum dot-stained cells. As shown in FIG. 5A, protein and PEG-coated quantum dots show a reduced level of non-specific binding in comparison with carboxylated dots. Surprisingly, the hydroxylated quantum dots of the present disclosure showed almost no non-specific binding in the microplate assays, in agreement with the fluorescence microscopy results shown in FIGS. 4A-4D. Additionally, non-specific quantum dot binding had an approximately linear relationship with the quantum dot concentration, a behavior that is consistent with non-specific interactions (FIG. 5B).

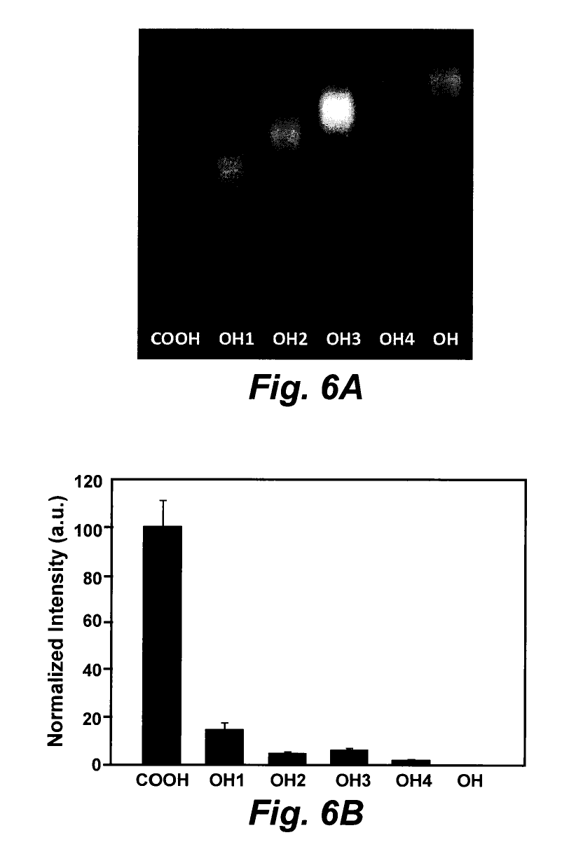

Surface Charge Variations. The degree of hydroxylation affects cellular non-specific binding, as shown with a series of quantum dot samples with increasing hydroxyl densities. As illustrated in FIG. 6A, the degree of hydroxylation was measured by gel electrophoresis, and its effect on the non-specific quantum dot binding was analyzed with fluorescence microplate assays, as shown in FIG. 6B.

Fully carboxylated quantum dots had the highest negative charge, and migrated the furthest. As the degree of hydroxylation was increased, the net surface charge was reduced, thereby retarding quantum dot migration in the gel. The migration distance plateaued as the degree of hydroxylation approached 100%. While not wishing to be bound by any one theory, this plateauing effect is possibly due to the increasing difficulty to hydroxylate the carboxylic acid groups due to steric hindrance. As expected, non-specific binding was dependent on the degree of hydroxylation, with fully hydroxylated quantum dots showing virtually no non-specific binding.

A number of different techniques are known in the art to protect the surface of semiconductor quantum dots (quantum dots) and render them soluble in water for biological applications. These techniques have generally fallen into two categories: (a) exchanging hydrophobic ligands on the quantum dot surface with hydrophilic ligands, or (b) coating the nanoparticles with an amphiphilic polymer that can interact with both the hydrophobic ligands and the external aqueous environment. While coating procedures that preserve the hydrophobic ligands show improved optical properties compared to ligand exchange procedures (Smith et al., (2006) Phys. Chem. Chem. Phys. 8: 3895-3903), the resulting quantum dots are large and do not necessarily perform well for complex samples such as cells and tissues.

The size of these nanoparticles can be further increased by surface treatments that are designed to reduce non-specific binding. These treatments generally involve attaching proteins (such as, but not limited to, streptavidin) or polyethylene glycol (PEG) to the quantum dot surface, and which have been shown to reduce, but not eliminate the non-specific binding. New coating methods to minimize non-specific binding while maintaining the small size and stability of the quantum dot probes are desirable. Nanoparticle surface charges were considered to play a critical role, because quantum dots with either highly negatively or positively charges show significant non-specific binding to cells and tissues (Pathak et al., (2001) J. Am. Chem. Soc. 123: 4103-4104; Gerion et al., (2001) J. Am. Chem. Soc. 124: 24; Bentzen et al., (2005) J. Bioconjugate Chem. 16: 1488-1494; Duan & Nie (2007) J. Am. Chem. Soc. 129: 3333-3338). Electrostatic interactions between proteins or other molecules play a significant role in molecular recognition, control of protein phosphorylation and the enhancement of catalytic rates (Honig & Nicholls (1995) Science 268: 1144-1149). These charged regions could interact with quantum dots electrostatically, leading to considerable non-specific binding as observed for highly charged quantum dots.

Methods of use: As mentioned above, the present disclosure relates generally to methods for detecting, localizing, and/or quantifying biological targets, cellular events, diagnostics, cancer and disease imaging, gene expression, protein studies and interactions, and the like. The present disclosure also relates to methods for multiplex imaging inside a host living cell, tissue, or organ, or a host living organism, using embodiments of the present disclosure. The present disclosure also relates to diagnosing the presence of diseases and cancer, treating diseases and cancer, monitoring the progress of diseases and cancer, and the like.

Multiplexed quantum dot probes according to the present disclosure are advantageous for molecular disease diagnosis. In particular, quantum dot probes of the present disclosure can be used to measure a panel of biomarkers in intact cancer cells and tissue specimens, allowing a correlation of traditional histopathology and molecular signatures. With minimized non-specific binding and background interference, the quantum dot probes of the present disclosure are especially suited for analyzing cancer biomarkers that are present at low concentrations or in small numbers of cells.

The biological target can include, but is not limited to, viruses, bacteria, cells, tissue, the vascular system, microorganisms, artificially constituted nanostructures (e.g., micelles), proteins, polypeptides, antibodies, antigens, aptamers (polypeptide and polynucleotide), polynucleotides, and the like, as well as those biological targets described in the definition section above.

Kits: This disclosure encompasses kits, which include, but are not limited to, coated quantum dots and directions (written instructions for their use). The components listed above can be tailored to the particular study to be undertaken. The kit can further include appropriate buffers and reagents known in the art for administering various combinations of the components listed above to the host cell or host organism.

The present disclosure provides a new class of hydroxylated quantum dots. The quantum dots have a hydroxylated coat disposed thereon that serves to minimize non-specific cellular binding while retaining the small size of quantum dot probes. They were prepared from carboxylated (—COOH) dots via a hydroxylation step. Optional cross-linking within the coating is also possible. The hydroxyl-coated dots have compact sizes of about 13 to about 14 nm hydrodynamic diameter, just slightly larger than the diameter of uncoated quantum dots, and are bright with about 65% quantum yields. They are also very stable under both basic and acidic conditions. Quantitative data from human cancer cells indicate that the hydroxylated quantum dots results in a significant (>100-fold) reduction in non-specific binding relative to that of carboxylated dots, and a smaller, but still significant, reduction relative to protein and PEG-coated dots. The data indicate that surface charge plays a significant role in the non-specific binding of these nanoparticles to cellular components. The nanoparticles of the disclosure are advantageous in a range of biological applications where non-specific binding is a major problem, such as in multiplexed biomarker staining in cells and tissues, detection of biomarkers in body fluid samples (blood, urine, etc.), as well as live cell imaging.

One aspect of the present disclosure provides a nanostructure, comprising: a quantum dot; a hydrophobic layer disposed on the quantum dot; and a coat disposed on said hydrophobic layer, wherein the coat has a substantially hydroxylated outer surface or a substantially zwitterion outer surface. The term “substantially” can describe something as greater than 50%, 60%, 70%, 80%, 90%, or 95%.

In preferred embodiments of the present disclosure, the nanostructure has a coating, wherein said coating has a substantially hydroxylated outer surface.

In embodiments of this aspect of the disclosure, the nanostructure may further comprise at least one layer selected from the group consisting of: a capping layer, a polymer layer, a target-specific probe layer, or any combination thereof.

In one embodiment of the disclosure, the nanostructure may have a hydrodynamic diameter of about 12 to about 15 nm.

In another embodiment, the nanostructure may have a hydrodynamic diameter of about 13 to about 14 nm.

In embodiments of the nanostructure according to the disclosure, the nanostructure may a zeta potential of about −17 to about −23 mV at pH of about 8.5.

In other embodiments, the nanostructure may have a zeta potential of about −19 to about −21 mV at pH of about 8.5.

In the various embodiments of the present disclosure, the nanostructure may have substantially no detectable non-specific cellular binding compared to a nanostructure not having a coat that is substantially hydroxylated or a substantially zwitterion outer surface and at the same concentration.

In one embodiment of the disclosure, the nanostructure has greater than about 60% quantum yield.

In other embodiments of the disclosure, the nanostructure may be stable under acidic and basic conditions.

In some embodiments, the nanostructure is stable under acidic conditions.

In other embodiments, the nanostructure is stable under basic conditions.

Another aspect of the disclosure provides methods of synthesizing a nanostructure, where the methods comprise: (a) providing a quantum dot, wherein the quantum dot comprises a hydrophobic layer thereon; (b) encapsulating the quantum dot by contacting the quantum dot with a polymer comprising a multiplicity of carboxyl groups; and (c) replacing a preponderance of the carboxyl groups with a multiplicity of hydroxyl groups or a multiplicity of zwitterions.

In preferred embodiments of the method of the present disclosure, the preponderance of the carboxyl groups may be replaced by a multiplicity of hydroxyl groups.

In some embodiments, the aliphatic chain of the poly(acrylic acid)-aliphatic amine polymer may be a C₄-C₁₈ aliphatic chain.

In other embodiments, the aliphatic chain of the poly(acrylic acid)-aliphatic amine polymer may be a C₁₂ aliphatic chain.

In one embodiment of the disclosure, the aliphatic chain of the poly(acrylic acid)-aliphatic amine polymer is a C₈ aliphatic chain.

In embodiments of the method of the present disclosure, step (c) may comprise contacting the quantum dot having the polymer coat thereon with a water soluble diimide, and an amine alcohol, thereby replacing a preponderance of the carboxyl groups of the multiplicity of carboxyl groups with a multiplicity of hydroxyl groups.

In these embodiments of the disclosure, the water soluble diimide may be selected from the group consisting of: 1-ethyl-3-(3-dimethylaminopropyl) carbodiimide (ECDI) and N-(3-dimethylaminopropyl)-N′-ethylcarbodiimide hydrochloride (EDAC).

In one embodiment of the disclosure, the water soluble diimide is N-(3-dimethylaminopropyl)-N′-ethylcarbodiimide hydrochloride (EDAC).

In the embodiments of the disclosure, the amine alcohol may be selected from the group consisting of: 1,3-diamino-2-propanol (DAP), ethanolamine, 3-amino-1-propanol, 3-amino-1,2-propanediol, 2-amino-1,3-propanediol (serinol), 4-amino-1-butanol, 2-(2-aminoethoxy)ethanol, Tris(hydroxymethyl)aminomentane, 1,4-diamino-2,3-butanediol, 5-amino-1-pentanol, 2-(3-aminopropylamino)ethanol, 6-amino-1-hexanol, and N,N-bis(2-hydroxyethyl)ethylenediamine.

In one embodiment, the amine alcohol is 1,3-amino-2-propanol (DAP), thereby replacing a preponderance of the carboxyl groups of the multiplicity of carboxyl groups with a multiplicity of hydroxyl groups.

In the embodiments of the method of the present disclosure, step (c) may further comprise contacting the quantum dot having the polymer coat thereon with N-hydroxysulfosuccinimide sodium salt (Sulfo-NHS).

In one embodiment of the method of the disclosure, step (c) comprises contacting the quantum dot having the polymer coat thereon with N-hydroxysulfosuccinimide sodium salt (Sulfo-NHS), N-(3-dimethylaminopropyl)-N′-ethylcarbodiimide hydrochloride (EDAC), and 1,3-amino-2-propanol (DAP), thereby replacing a preponderance of the carboxyl groups of the multiplicity of carboxyl groups with a multiplicity of hydroxyl groups.

In other embodiments of the disclosure, step (c) may comprise contacting the quantum dot having the polymer coat thereon with a water soluble diimide, and an alkylamine, thereby replacing a preponderance of the carboxyl groups of the multiplicity of carboxyl groups with a multiplicity of zwitterions.

In these embodiments of the disclosure, the alkylamine may be selected from the group consisting of: (2-aminoethyl)trimethylammonium chloride hydrochloride, n,n-dimethylethylenediamine, 3-(dimethylamino)-1-propylamine, 2-(aminomethyl)-2-methyl-1,3-propanediamine trihydrochloride, n-(2-aminoethyl)-1,3-propanediamine, and 3,3′-diamino-N-methyldipropylamine.

Yet another aspect of the disclosure provides methods imaging, comprising: providing a nanostructure of the present disclosure; administering the nanostructure to a recipient host; and imaging the recipient host, whereby the nanostructure delivered to the recipient host provide an image of a tissue of the recipient host, and wherein the image has a substantially reduced non-tissue-specific background fluorescence when compared to an image generated with a nanostructure not having a substantially hydroxylated coat.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. The term “substantially” can describe something as greater than 50%, 60%, 70%, 80%, 90%, or 95%. The term “preponderance” can describe something as greater than 50%, 60%, 70%, 80%, 90%, or 95%. The term “multiplicity” can include greater than 1.

### EXAMPLES

### Example 1

QD Synthesis and Encapsulation. CdSe/CdS/ZnS quantum dots were synthesized using previously described methods (Qu & Peng (2002) J. Am. Chem. Soc. 124: 2049-2055; Smith et al., (2006) Phys. Chem. Chem. Phys, 8: 3895-3903, both of which are incorporated herein by reference in their entireties). Before surface encapsulation, the quantum dots were precipitated with acetone to remove excess octadecylamine, and were redispersed in chloroform. Encapsulation was carried out by mixing 2 mg of poly(acrylic acid)-octylamine polymer and 1 nmol of quantum dots in chloroform The mixture was vortexed for 5 min and the solvent was removed under vacuum. The dried film was dissolved in 50 mM borate buffer, sonicated for 15 min and centrifuged at 6000 g for 15 min to yield a clear supernatant. The free polymer was removed by dialyzing the supernatant against 50 mM borate buffer.

### Example 2

Surface Hydroxylation and Crosslinking. Purified quantum dots were diluted in deoinized water for surface modification. Briefly, 100 pmol of quantum dots was diluted to a final concentration of 100 nM. Approximately 1 mg of N-hydroxysulfosuccinimide sodium salt (Sulfo-NHS) and 12 mg N-(3-Dimethylaminopropyl)-N′-ethylcarbodiimide hydrochloride (EDAC) were added to the quantum dot solution and were mixed thoroughly. For hydroxylation and cross-linking, 11.5 mg of 1,3-amino-2-propanol (DAP) was dissolved in deionized water and added slowly to the quantum dot solution under vigorous stirring. The mixture was allowed to react for 2 hours and was then dialyzed against 50 mM borate buffer to remove excess reactants and byproducts. To vary the degree of hydroxylation, EDAC and DAP amounts were decreased appropriately.

### Example 3

Gel Electrophoresis. Quantum dots were evaluated with a horizontal submerged gel electrophoresis apparatus (Mini-SubCell GT, BIO-RAD) using a 0.7% (w/v) agarose gel in Tris-acetate-EDTA (TAE) buffer. Briefly, a 250 mL beaker was charged with 0.35 g of agarose, to which 50 mL of 1×TAE buffer at pH 8.5 was added. The solution was then covered with a 50 mL beaker and heated in a microwave until completely melted, approximately 1 minute. The molten agarose was allowed to stand at room temperature for 10 minutes, at which point 50 μL of Tween-20 was added for a final concentration of 0.01% (v/v). The solution, when at about 55° C., was cast into a gel tray with a 1.0 mm 15 well comb and allowed to solidify. The gel was placed in the agarose electrophoresis tank and sufficient 1×TAE buffer was added to the tank to just cover the top of the gel. For each well, 20 μL of the quantum dot samples at 100 nM were mixed with 5 μL of 5×TAE loading buffer (5×TAE, 25% (v/v) glycerol, 0.25% (w/v) Orange-G at pH 8.5) by pipetting before being loaded into the gel. The gel was resolved at 100 V for 30 minutes (PowerPak Basic, BIO-RAD) and then imaged with 2-second exposure using a UVP gel documentation system.

### Example 4

Cell Fixing and Staining. Hela cells (ATCC number CCL-2) were cultured in RPMI media with 10% fetal bovine serum (FBS) at 37° C. (5% CO₂) and grown in an 8-well chamber slide. After 24 hours for seeding, the cells were washed with 1×PBS and fixed with 3.7% formaldehyde and 0.1% triton X in 1×PBS for 5 minutes. The fixative was then aspirated and the cells washed with 1×PBS 3 times for 5 min each. A 2% BSA blocking solution in 1×PBS was added to the wells for 20 min and then aspirated. Quantum dots were diluted in the blocking solution to the desired concentration and incubated with the cells for 20 min. The quantum dot staining solution was aspirated and the cells were washed with 1×PBS 3 times for 5 min each. A 1 μg/mL solution of 4′,6-diamidino-2-phenylindole dihydrochloride (DAPI) in deionized water was added to the wells and incubated for 5 min for nuclear staining. The DAPI solution was then aspirated and the cells were washed for 5 min with deionized water. The slide was mounted and prepared for fluorescence microscopy.

### Example 5

QD Binding Assays. For quantitative analysis of non-specific quantum dot binding to cells, a fluorescent microplate reader (Synergy 2 Multi-detection Microplate Reader, Biotek Instruments) was used. Briefly, HeLa cells were cultured in a clear bottom 96 well plate for 24 hours, fixed, blocked and stained as described in Example 4. DAPI nuclear staining was performed to correct for variations in cell densities. Quantum dot concentrations from 0 to 20 nM were used in replicates of six (n=6). The fluorescence intensity from the quantum dots was measured, normalized using the DAPI fluorescence intensity, and background subtracted to determine the intensity of non-specific staining.

We claim:

1. A nanostructure, comprising:
a quantum dot;
a hydrophobic layer disposed on the quantum dot; and
a coat disposed on said hydrophobic layer, wherein the coat has a substantially hydroxylated outer surface or a substantially zwitterion outer surface.

2. The nanostructure according to claim 1, wherein the coat has a substantially hydroxylated outer surface.

3. The nanostructure according to claim 1, wherein the nanostructure further comprises at least one layer selected from the group consisting of: a capping layer, a polymer layer, a target-specific probe layer, and a combination thereof.

4. The nanostructure according to claim 1, wherein the nanostructure has a hydrodynamic diameter of about 12 to about 15 nm.

5. The nanostructure according to claim 1, wherein the nanostructure has a hydrodynamic diameter of about 13 to about 14 nm.

6. The nanostructure according to claim 1, wherein the nanostructure has a zeta potential of about −17 to about −23 mV at pH of about 8.5.

7. The nanostructure according to claim 1, wherein the nanostructure has a zeta potential of about −19 to about −21 mV at pH of about 8.5.

8. The nanostructure according to claim 1, wherein the nanostructure has substantially no detectable non-specific cellular binding compared to a nanostructure not having a coat that is substantially hydroxylated or having a substantially zwitterion outer surface, and at the same concentration.

9. The nanostructure according to claim 1, wherein the nanostructure has greater than about 60% quantum yield.

10. The nanostructure according to claim 1, wherein the nanostructure is stable under acidic and basic conditions.

11. The nanostructure according to claim 1, wherein the nanostructure is stable under acidic conditions.

12. The nanostructure according to claim 1, wherein the nanostructure is stable under basic conditions.

13. A method of synthesizing a nanostructure comprising:
(a) providing a quantum dot, wherein the quantum dot comprises a hydrophobic layer thereon;
(b) encapsulating the quantum dot by contacting the quantum dot with a polymer comprising a multiplicity of carboxyl groups; and
(c) replacing a preponderance of the carboxyl groups by a multiplicity of hydroxyl groups or a multiplicity of zwitterions.

14. The method according to claim 13, wherein the preponderance of the carboxyl groups are replaced by a multiplicity of hydroxyl groups.

15. The method according to claim 13, wherein the aliphatic chain of the poly(acrylic acid)-aliphatic amine polymer is a C₄-C₁₈ aliphatic chain.

16. The method according to claim 13, wherein the aliphatic chain of the poly(acrylic acid)-aliphatic amine polymer is a C₁₂ aliphatic chain.

17. The method according to claim 13, wherein the aliphatic chain of the poly(acrylic acid)-aliphatic amine polymer is a C₈ aliphatic chain.

18. The method according to claim 13, wherein step (c) comprises contacting the quantum dot having the polymer coat thereon with a water soluble diimide, and an amine alcohol, thereby replacing a preponderance of the carboxyl groups of the multiplicity of carboxyl groups with a multiplicity of hydroxyl groups.

19. The method according to claim 18, wherein the water soluble diimide is selected from the group consisting of: 1-ethyl-3-(3-dimethylaminopropyl) carbodiimide (ECDI) and N-(3-dimethylaminopropyl)-N′-ethylcarbodiimide hydrochloride (EDAC).

20. The method according to claim 18, wherein the water soluble diimide is N-(3-dimethylaminopropyl)-N′-ethylcarbodiimide hydrochloride (EDAC).

21. The method according to claim 13, wherein the amine alcohol is selected from the group consisting of: 1,3-diamino-2-propanol (DAP), ethanolamine, 3-amino-1-propanol, 3-amino-1,2-propanediol, 2-amino-1,3-propanediol (serinol), 4-amino-1-butanol, 2-(2-aminoethoxy)ethanol, Tris(hydroxymethyl)aminomentane, 1,4-diamino-2,3-butanediol, 5-amino-1-pentanol, 2-(3-aminopropylamino)ethanol, 6-amino-1-hexanol, and N,N-bis(2-hydroxyethyl)ethylenediamine.

22. The method according to claim 13, wherein the amine alcohol is 1,3-amino-2-propanol (DAP), thereby replacing a preponderance of the carboxyl groups of the multiplicity of carboxyl groups with a multiplicity of hydroxyl groups.

23. The method according to claim 13, wherein step (c) further comprises contacting the quantum dot having the polymer coat thereon with N-hydroxysulfosuccinimide sodium salt (Sulfo-NHS).

24. The method according to claim 13, wherein step (c) comprises contacting the quantum dot having the polymer coat thereon with N-hydroxysulfosuccinimide sodium salt (Sulfo-NHS), N-(3-dimethylaminopropyl)-N′-ethylcarbodiimide hydrochloride (EDAC), and 1,3-amino-2-propanol (DAP), thereby replacing a preponderance of the carboxyl groups of the multiplicity of carboxyl groups with a multiplicity of hydroxyl groups.

25. The method according to claim 13, wherein step (c) comprises contacting the quantum dot having the polymer coat thereon with a water soluble diimide, and an alkylamine, thereby replacing a preponderance of the carboxyl groups of the multiplicity of carboxyl groups with a multiplicity of zwitterions.

26. The method according to claim 25, wherein the alkylamine is selected from the group consisting of: (2-aminoethyl)trimethylammonium chloride hydrochloride, n,n-dimethylethylenediamine, 3-(dimethylamino)-1-propylamine, 2-(aminomethyl)-2-methyl-1,3-propanediamine trihydrochloride, n-(2-aminoethyl)-1,3-propanediamine, and 3,3′-diamino-N-methyldipropylamine.

27. A method of imaging, comprising:
providing a nanostructure according to claim 1;
administering the nanostructure to a recipient host; and
imaging the recipient host, whereby the nanostructure delivered to the recipient host provide an image of a tissue of the recipient host, and wherein the image has a substantially reduced non-tissue-specific background fluorescence when compared to an image generated with a nanostructure not having a substantially hydroxylated coat.
